(19) United States
(12) Patent Application Publication
(10) Pub. No.: US 2025/0285720 A1
(43) Pub. Date: Sep. 11, 2025

(54) Filtering System for Managing the Treatment of a Patient

(71) Applicant: GAIA AG, Hamburg (DE)

(72) Inventors: Bernhard Wellhöfer, Hamburg (DE); Mario Weiss, Hamburg (DE)

(21) Appl. No.: 18/601,489

(22) Filed: Mar. 11, 2024

(51) Int. Cl. G16H 20/00; G16H 10/65

(52) U.S. Cl. CPC G16H 20/00; G16H 10/65

(57) ABSTRACT

A system for managing treatment of a patient is provided with a computer in data communication with a database storing a plurality of prompts, medical content, and patient medical history. A user device in data communication with the computer presents a first prompt to the patient and receives the patient reaction to the first prompt. Software executing on the computer parses the patient reaction into discrete and non-discrete portions, with a generative artificial intelligence program parsing the non-discrete portions to extract a key data set. A determinative artificial intelligence program then correlates the key data set and discrete response portions with the content in the database to create a determinative data set. A generative artificial intelligence program receives the determinative data set and non-discrete portions of the reaction and generates a second prompt to be displayed to the patient to refine their treatment.

### TECHNICAL FIELD

The present disclosure relates to a novel system for managing the treatment of a patient. More specifically, the present disclosure relates to using a combination of a generative artificial intelligence program and a determinative artificial intelligence program to reliably and repeatably receive and deliver medical content to and from a patient while also tailoring such medical content to the specific patient presented to the system.

### BACKGROUND

Medical diagnosis and treatment, and the management thereof, is a rules-based practice driven by clear guidelines on what recommendations to make, what drugs to prescribe, and other such actions dependent on specific conditions of the patient in question. This can be considered the technical, scientific, or evidence-based part of medical diagnosis and treatment and the management thereof.

At the same time, medical diagnosis and treatment, and the management thereof, is highly individual and must address patient-specific questions and circumstances in the interaction with the patient. This can be considered the empathic or human part of medical diagnosis and treatment and the management thereof.

Both the evidence-based and empathic elements are intertwined with one another in the day-to-day practice of medicine.

Presently, there exist deterministic artificial intelligence systems (e.g., an expert system) which can parse and hold medical knowledge. Such deterministic artificial intelligence systems are mostly rules-based and represent the evidence-based knowledge of medicine. These systems are deterministic, and as a consequence the results of these systems are controllable, comprehensible, and repeatable. This is highly beneficial in that deterministic thinking is crucial in the evidence-based element of the practice of medicine. Information such as drug dosage must always be provided in a controlled manner with no ambiguity and high degrees of repeatability or reliability. However, such a system on its own does not adapt well to external circumstances such as a patient's life situation, feelings, and questions.

Presently, there exist generative artificial intelligence systems (e.g., an LLM AI) which can also parse and hold medical knowledge. Such generative artificial intelligence systems are not deterministic and not repeatable or predictable. At the same time, these generative artificial intelligence systems can generate tailored interaction to high degrees of specificity to a given patient and include the degrees of flexibility necessary to interpret patient requests and vague (e.g., lacking a correct answer or a measurable element) external circumstances such as a patient's life situation, feelings, and questions.

### SUMMARY

For these and other reasons known to a person of ordinary skill in the art, what is needed is a system that allows for a combination of repeatable and reliable in-depth medical knowledge with the need for flexibility and interpretation of a specific patient and their needs.

A goal of the present disclosure is to provide a system that can incorporate an unlimited amount of rules and can easily be kept up-to-date, having knowledge broader, deeper, and newer than that of a human physician.

Another goal of the present disclosure is to provide a system with a degree of flexibility and which may be programmed to simulate empathy in the psychosocial context of the patient.

Another goal of the present disclosure is to provide a system with no sociocultural bias and which does not rely solely on prior scenarios in which the exact system has been deployed.

Another goal of the present disclosure is to provide a system which can provide psychological interventions, such as fear reduction techniques or interventions which reduce depressive thoughts (e.g., where a patient may have a long-term terminal condition such as cancer), which generally are not provided by specialized physicians not trained in other fields.

Another goal of the present disclosure is to provide a system that may mimic a well-educated scientifically oriented medical expert and further vary that expertise in a tailored manner to the specific context of the patient, while retaining a highly controlled and scalable process.

Another goal of the present disclosure is to provide a system that can effectively track a patient over the patient's lifetime (e.g., in the case of a life-long illness) which typically cannot be accomplished by a human physician who may move, retire, change specialties, or other such interruptions in continuity of care.

Another goal of the present disclosure is to provide a system that can receive feedback to better understand patient feedback including emotional states, physical states, and other states.

Another goal of the present disclosure is to provide a system that can signal third parties in an emergency.

In one aspect of the present invention, a system for managing treatment of a patient is provided with a computer and a database in data communication with said computer. The database has a plurality of prompts and a plurality of medical content and a plurality of patient medical history. A user device is also in data communication with said computer for presenting a first prompt received from said computer to the patient. A life monitoring system is also in data communication with said computer with said life monitoring system observing aspects of the patient and relaying that biometric data information to said computer. The user device receives a first reaction of the patient to the first prompt. The first reaction may be in the form of a text response (e.g., the patient may type out words for submission), a selection (e.g., the patient may select one or more of A, B, and C, or the patient may select a position on a scale indicating a degree of severity), a voice response, and patient biometric data from the life monitoring system. Software executing on the computer receives the first reaction from the patient, and may pass that information on to the database as a recorded entry or the database may directly receive that information. Software executing on the computer configured with a generative artificial intelligence program parses the first patient reaction to generate a first key data set. Software executing on the computer configured with a determinative artificial intelligence program receives the first key data set and accesses the database to associate one or more elements of the first key data set with one or more of the plurality of content in the database, thus generating a first determinative data set. Software executing on the computer configured with the generative artificial intelligence program receives the first determinative data set, and generates a second prompt associated with medical content. The user device then presents the second prompt to the patient. This process may be generally repeated by the system.

In another aspect of the present invention, the system for managing treatment of a patient may further comprise software executing on the computer for training a machine learning algorithm based on at least one of the patient medical history, medical content, and patient reaction(s). The software executing on the computer configured with the determinative artificial intelligence program may identify any discrete portions of the first reaction of the patient, and receive those discrete portions of the first reaction before the first reaction is parsed by the software executing on the computer configured with the generative artificial intelligence program. Those non-discrete portions may be one of an image, text, or audio response (e.g., reactions or responses which may have ambiguity or require interpretation). Software executing on the computer may further be configured to determine relevant medical data associated with the first reaction of the patient, and the software executing on the computer configured with the generative artificial intelligence program may modify the generated second prompt (and the associated medical content included therein) in response to the relevant medical data. The software executing on the computer configured with the generative artificial intelligence program may be configured to introduce variance into the second prompt (e.g., given the same inputs, the output may not be identical in form, style, or other similar metric). The software executing on the computer configured with the determinative artificial intelligence program may be configured to introduce no variance into the second prompt when associating one or more elements of the first key data set with one or more of the plurality of prompts and medical content in the database, thereby generating substantially the same determinative data set from the same (or proximately the same) reaction of the patient.

In yet another aspect of the present invention, the system for managing treatment of a patient may further comprise storing the medical history of the specific patient in question in the database. Such medical history may include patient reactions and system generated prompts from prior visits. Software executing on the computer configured with the generative artificial intelligence program may associate historical (e.g., from past interactions) patient reactions to historical system generated prompts, and further introduce variance into the first and second prompts to elicit improved patient reactions.

### BRIEF DESCRIPTION OF THE DRAWINGS

FIG. 1 shows a schematic diagram of the presently disclosed system.

FIG. 2 shows a schematic diagram of the presently disclosed system with additional elements.

FIG. 3 shows a simplified schematic diagram of the presently disclosed system.

### DETAILED DESCRIPTION

The present invention will now be described by referencing the appended figure.

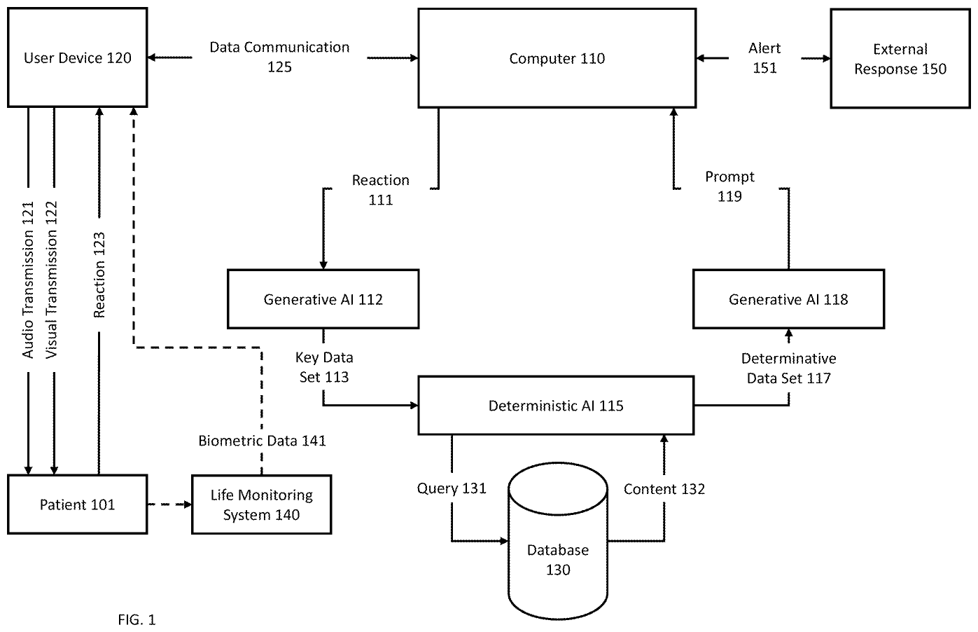

Referring to FIG. 1, the present disclosure describes a system 100 for managing treatment of a patient.

The system 100 includes a computer 110. The computer 110 may be a processor, remote computer, computer server, network, or any other computing resource, including mobile devices.

The computer 110 may be in data communication 125 with a user device 120. The user device 120 may be a computer, laptop, smartphone, tablet, or other electronic device, including mobile devices, capable of transmitting data to the computer 110. The user device 120 may generally interact with and be manipulable by the patient. User device 120 may run an application on a mobile device or smartphone. The user device 120 may have an input device such as a mouse and keyboard, touchscreen, trackpad, etc. The user device 120 may include a display. Data communication 125 may function in both directions, allowing the user device 120 and computer 110 to pass information back and forth.

The user device 120 may include at least one sensor or may be in communication with at least one sensor, where the user device 120 is capable of and configured to receive data inputs from an external supplemental system. The sensor may be a life monitoring system 140, capable of and configured to collect biometric data 141 from the patient and transmit said biometric data 141 to the user device 120. The life monitoring system 140 may include a camera or the user device 120 may include an integrated camera or otherwise be in communication with one. The camera may be a webcam, still camera, video camera, etc. The biometric data may be any measurable vital aspect related to the life or health of the patient including weight, body temperature, heart rate, blood pressure, blood sugar level, pupil dilation, and visual images of the patient or detailed images of portions of the patient's body. Visual images of the patient may be used to approximate or measure certain aspects of a patient such as their fear level, physical manifestations of psychological concerns such as sweating or nervousness or twitching, the time it takes the patient to respond or react to a prompt and any hesitation they exhibit in making that decision, and other aspects.

The computer 110 may also be in communication with a database 130. The database 130 may be a storage drive or array accessible to computer 110, or cloud storage. Information within the database includes the medical history of the patient before the system, a plurality of patient medical history of any patient who has ever been before the system or the patient medical history of other patients which the system has access to, a plurality of medical information which includes medical texts and journals or articles, current and past valid treatment guidelines, study data and text, and a plurality of prompts. The plurality of patient medical history may include anonymized medical history of other patients who are not the specific patient in question, though the anonymization process will preferably retain internal correlations and relations to the various records for a given patient. Prompts include those presented to the patient (121 and 122) and also prompts generated by the system 119 and historical prompts previously presented to the patient in question and other patients stored in the database 130. Prompts in the database 130 may be indexed or searchable by queries 131 and the system will index those prompts by a number of different identifiers as disclosed herein (e.g., the height or weight of the patient the prompt was presented to, the symptoms bringing the patient before the system, and so on).

The computer 110 may be configured to run software capable of serving as a deterministic artificial intelligence program 115 and separately configured to also run software capable of serving as a generative artificial intelligence program 112. The generative 112 and determinative intelligence programs 115 may be hosted on independent servers or data centers with the large amount of computing power necessary to quickly generate responses in limited periods of computing time. In other embodiments, the computer 110 may be a powerful computing device but not so large as to require its own building like, e.g., a data center. Here, the generative 112 and determinative intelligence programs 115 may be interoperable modules in direct (e.g., hard-wired graphics cards or equivalent removeable modules which may contain relatively self-sufficient computing systems to operate the intelligence programs) data communication with the computer 110. In some embodiments, both the deterministic 115 and generative 112 may be capable of being performed by a single, unitary software program. Generally, such a configuration may be difficult or impossible where the computer 110 and user device 120 are a single, unitary item, e.g., where the system is operating solely on a tablet computer or another type of mobile device with limited computing power. In such a configuration and in other configurations, it may be preferable to provide data communication between the computer/user device 110/120 combination and a server or data center where the artificial intelligence program is operating. The computer 110 would then send and receive individual requests to and from the artificial intelligence programs 112, 115 operating in a remote environment, and the computer 110 may be in data communication with a database 130, such that the computer 110 is acting as a hub of communication and sending and receiving data which are generated outside of the computer.

The database 130 may store information regarding the system 100, including a plurality of medical content and a plurality of patient medical history and a plurality of prompts. The information stored in the database 130 may be accessed by query 131 provided by the patient 101 via reaction 123. The information stored in the database 130 may also be accessed by query 131 generated by the deterministic artificial intelligence program 115 software operating on the computer 110 configured to do so. These queries 131 may be based in part or in whole on the parsed patient reaction 111 and the key data set 113. Content of all types within the database 130 may be kept up-to-date on a live basis, for example via an internet connection or operator entry, or the content may be updated on a periodic basis, for example when new issues of diagnostic manuals are released or when research is published. The plurality of medical content in the database 130 may include, for example, established up-to-date medical procedures, diagnostic flow charts, clinical guidelines and algorithms, differential diagnosis tools, decision support systems, medical checklists or pathways in patient care in combination with structured methods for generating correlation of patient symptom(s) in relation to plurality of medical content in database 130. Methods for generating correlation of patient symptom(s) in relation to database 130 content include, for example, cross referencing symptom(s) with diagnostic flow charts or guidelines and medical publications describing diseases, implementation of machine learning including implementations of such learning on anonymized historical patient symptoms and diagnosis, natural language processing, clustering and pattern recognition, and predictive models or feedback loops for offering detailed diagnostic decision-making through a series of logical steps. The plurality of patient medical history may be tied to a specific patient 101 interacting with the system 100 presently, and may include the specific patient's prior medical history of all types (e.g., childhood vaccinations, past illnesses and medications either related or unrelated to a reason or symptom the specific patient is interacting with the system presently), and the specific patient's prior interactions with the system 100 (e.g., the types and content of previous recommendations or treatments provided to the patient and the patient's reaction to and willingness to follow the same, the number of times and frequency of prior interactions, the patient's satisfaction with prior interactions).

The plurality of patient medical history may include anonymized medical history of other patients who are not the specific patient in question, which may be useful, for example, in identifying the types of symptoms, treatments, and patient responses or reactions which may be similar or predictive of the specific patient 101 in question. For example, a specific patient 101 may have a persistent high blood pressure issue. The computer 110 may present to the patient a prompt in the form of audio 121 and visual 122 (e.g., text, images, and other visual content) content, to which the patient may respond 123 that the issue persists despite medication. The key data set 113 received by the deterministic artificial intelligence program 115 may include the patient's 101 age, weight, and gender. A query 131 to the database 130 may seek to match the general age, weight, and gender of the specific patient 101 to other anonymized patients of similar condition and report back what types of other interventions were successful and not successful on the anonymized patients of similar condition. This information may be included in a determinative data set 117, which the generative artificial intelligence program may turn into a natural language prompt 119 for display to the specific patient 101. The system 100 may further prompt the patient 101 for additional information contained in the determinative data set 117 derived from content 132 in an attempt to further filter a treatment recommendation, e.g., does the patient live in a suburban area (where long walks may be possible) or urban area (where gyms or other fitness facilities may be in close proximity). The system 100 may further iterate this process during the course of the instant visit or across a series of visits. The system 100 may further monitor changes in the patient's 101 residence, e.g., did symptoms worsen following a move from an urban area (where more walking occurred) to a suburban area (where more driving occurred at the expense of physical activity).

Queries 131 may seek a category of prompts, or a specific prompt. Categories of prompts may depend on the prompt itself, the type of prompt (image, sound, etc.), previous prompts or the patient's reaction thereto, or categorizations based on conditions, medications, or other factors.

The computer 110 may further include software to determine the relevant emotion that a patient is experiencing. To that end, it may send a query 131 to the database 130 for a prompt or other content 132.

The user device 120 presents the prompt 119 to the patient 101 in the form of audio transmissions 121 and visual transmissions 122 (which may be text, videos, pictures, or other such content). The patient 101 may provide a response or reaction 123 in view of being presented with the prompt. In other cases, the patient 101 reaction 123 may be inferred by biometric data 141 collected by a life monitoring system 140. For instance, the patient's 101 facial expression may change. A camera integrated with or connected to the user device 120 may capture images of the patient's 101 facial expressions before, during, and after the patient is presented with the prompt. In other instances, the images may be captured by the life monitoring system 140 directly. Other types of biometric data 141 may similarly accomplish the same goal, for example by monitoring heart rate as a proxy for patient 101 stress or concern and by observing physical symptoms of a patient 101 that they either may not be aware of or as verification of same (e.g., where a patient as a neuromuscular degenerative disease biometric data 141 may indicate an improvement or decline in condition based on observable patient 101 tremors).

The software may use specialized generative and determinative artificial intelligence programs. These may include, for example, generative basis artificial intelligence programs undergoing fine-tuning over time to adapt to evolving medical standards and patient data, domain-specific artificial intelligence programs imbued with expert knowledge for precise medical insights, and explainable artificial intelligence models providing chain of reasoning or federative learning allowing customization of the artificial intelligence programs by the physician.

The computer may use third-party data stored in the database 130 to help determine the most effective prompt 119 which can be generated. Third party data may include the weather at location of patient 101, potential stressors such as crime rate (communicated crime in media), pollution, traffic (time spend in traffic), and psycho economics such as stock price, inflation rate, employment rate (specifically in the sector patient 101 is working in), and consumer index. For example, the system may weigh reactions 123 or biometric data 141 differently if it is a sunny versus a rainy day, or if stock prices are up or down.

The system 200 may run in the background and not interfere with other treatments or activities. In such situations, the system 200 may constantly refine its determination of the reaction 223 of the patient 201. The patient 201 may be aware of the system's determination and provide further input 223 confirming or correcting the system's 200 determination.

If a determined patient reaction 223 is potentially harmful, the computer 210 may generate an emergency alert 151. The alert may be communicated to an external response system 150 such as an EMS system, a designated contract, a medical professional, or other person or institution.

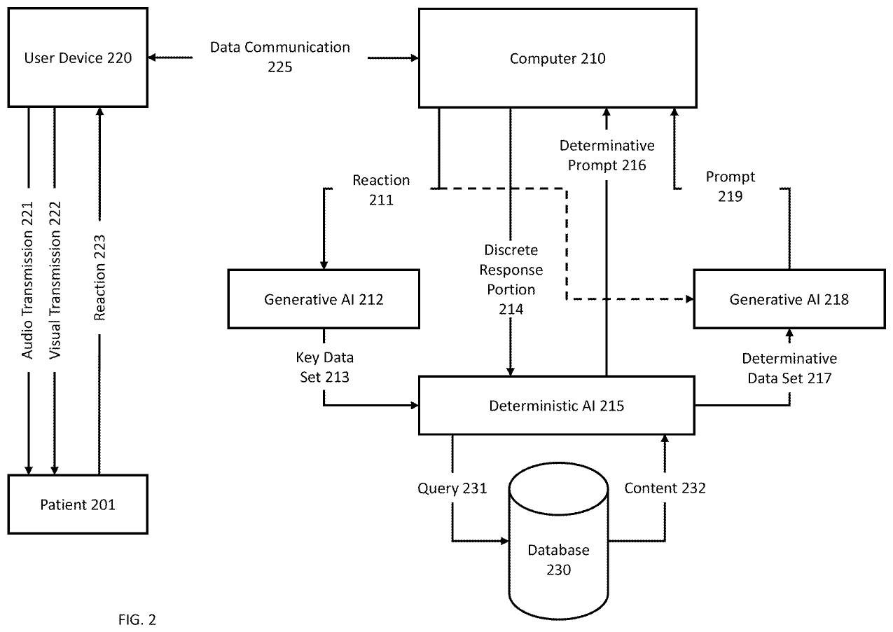

Referring to FIG. 2, the present disclosure describes a system 200 for managing treatment of a patient.

The system 200 is largely similar in form and function as the above system 100. However, in this embodiment, the system for managing treatment of a patient may further comprise software executing on the computer 210 for training a machine learning algorithm based on at least one of the patient medical history, medical content, and patient reaction(s). The software executing on the computer configured with the determinative artificial intelligence program 215 may identify any discrete portions 214 of the first reaction 223 of the patient and receive those discrete portions of the first reaction 214 before the first reaction 223 is parsed by the software executing on the computer configured with the generative artificial intelligence program 211. Non-discrete portions 211 may be identified by the software executing on the computer configured with the generative artificial intelligence program 212 and may be further parsed to extract deterministic data which is sent as key data set 213 to the software executing on the computer configured with the deterministic artificial intelligence program 215. Such non-discrete portions 210 may be one of an image, text, or audio response (e.g., reactions or responses which may have ambiguity or require interpretation). For example, non-discrete portions 211 which may be parsed to create a key data set 213 of discrete data may include a statement by the patient that “they don't feel so hot.” Discrete data from that statement may prove difficult to create, e.g., it may literally indicate that a patient does not have a fever (“is not hot”), but when parsed may create discrete data, e.g., that the patient is exhibiting worse symptoms than a prior visit.

Software executing on the computer 210 may further be configured to determine relevant medical data in the database 230 associated with the first reaction 223 of the patient, and the software executing on the computer configured with the generative artificial intelligence program 218 may modify the generated second prompt 219 (and the associated medical content 232 included therein) in response to the relevant medical data 232. The software executing on the computer configured with the generative artificial intelligence program may be configured to introduce variance into the second prompt (e.g., given the same inputs, the output may not be identical in form, style, or other similar metric). The software executing on the computer configured with the determinative artificial intelligence program may be configured to introduce no variance into the second prompt when associating one or more elements of the first key data set with one or more of the plurality of prompts and medical content in the database, thereby generating substantially the same determinative data set from the same (or proximately the same) reaction of the patient.

Software executing on the computer 210 may further be configured to determine relevant medical data in the database 230 associated with the first reaction 223 of the patient, and the software executing on the computer configured with the generative artificial intelligence program 218 may modify the generated second prompt 219 (and the associated medical content 232 included therein) in response to the non-discrete portions of the first reaction 211. The system 200 for managing treatment of a patient may further comprise storing the medical history of the specific patient in question in the database 230. Such medical history may include patient reactions and system generated prompts from prior visits. Software executing on the computer configured with the generative artificial intelligence program 212, 218 may associate historical (e.g., from past interactions) patient reactions to historical system generated prompts, and further introduce variance into the first and second prompts 219 to elicit improved patient reactions. The software executing on the computer configured with the generative artificial intelligence program 212, 218 may correlate particular content from a prior patient 201 interaction which elicited a recalled memory of symptoms or treatments and better manage treatment. For example, a patient 201 in a previous interaction with the system 200 may have mentioned that a particular activity was difficult to perform in the past. The same patient 201 may have difficulty articulating or remembering general body movements which were difficult in the past, and so the prompt 219 may be varied to re-frame the prompt 219 in the context of that particular activity, e.g., the prompt 219 may ask the patient 201 “whether it was still difficult to play tennis” or “whether jars were still difficult to open.” In other scenarios, the prompt 219 may include references to things that the patient 201 finds personally important or enjoyable, e.g., a prompt 219 may ask the patient 201 “how is your cat” where the database 230 contains previous interactions with the patient 201 where mention of the patient's 201 pets elicited improved disclosures in the reaction 223.

The first prompt presented to the patient 201 may incorporate content 232 from the database 230. Upon contact with a patient 201, the user device 220 may identify the patient 201 and pass this discrete information 214 to the computer 210 configured with the deterministic artificial intelligence program 215. The database 230 may be queried 231 with the patient 201 identify, and content 232 may be returned indicating historical prompts previously presented to the patient and other relevant information such as the reason a patient is engaging in interaction with the system 200 (e.g., the patient asked to be seen because of a skin condition, or a routine pre-scheduled visit such as an annual checkup). This determinative prompt information 216 may be passed directly to the patient 201, e.g., as a confirmation that the information is correct. This content 232 may further be incorporated into a determinative data set 217 which may form the basis of a prompt 219 in a natural language generated by the generative artificial intelligence program 218. A prompt 219 may further include any media that stimulates the patient 201. For example, a prompt 219 may include text, images, sound, video, physical stimuli, tasting material, etc.

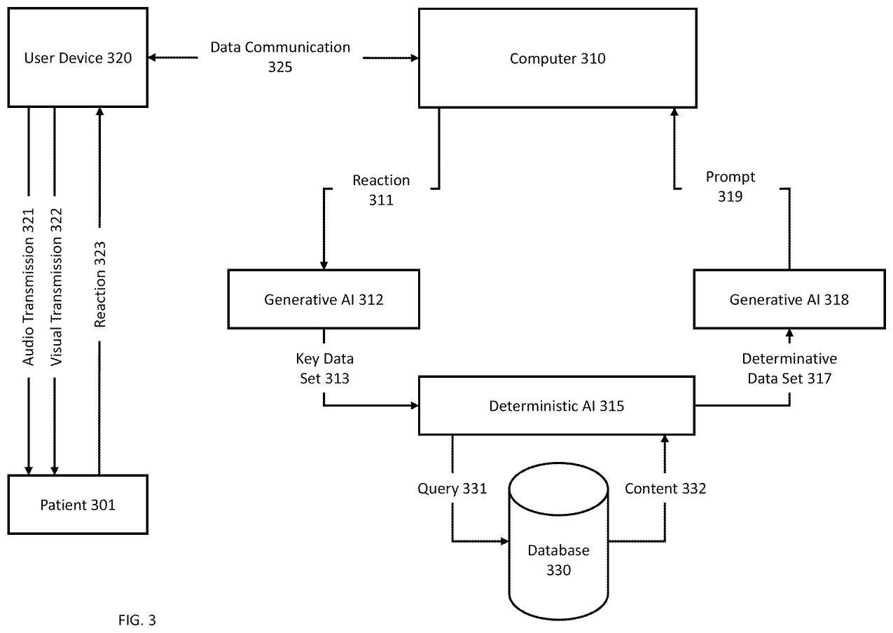

Referring to FIG. 3, the present disclosure describes a simplified system 300 for managing treatment of a patient.

Although the invention has been illustrated and described herein with reference to a preferred embodiment and a specific example thereof, it will be readily apparent to those of ordinary skill that the art that other embodiments and examples may perform similar functions and/or achieve user experiences. All such equivalent embodiments and examples are within the spirit and scope of the present invention, are contemplated thereby, and are intended to be covered by the following claims.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect. The present disclosure is to be considered as an example of the invention and is not intended to limit the invention to a specific embodiment illustrated by the figures above or description below.

For purposes of explanation and not limitation, specific details are set forth such as particular architectures, interfaces, techniques, etc., in order to provide a thorough understanding. In other instances, detailed descriptions of well-known devices, circuits, and methods are omitted so as not to obscure the description with unnecessary detail.

Generally, all terms used in the claims are to be interpreted according to their ordinary meaning in the technical field, unless explicitly defined otherwise herein. All references to a/an/the element, apparatus, component, means, step, etc., are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc., for different features/components of the present disclosure are only intended to distinguish the features/components from other similar features/components and not to impart any order or hierarchy to the features/components. As used herein, the term “and/or” includes any and all combinations of one or more of the associated listed items. As used herein, the term “application” is intended to be interchangeable with the term “invention”, unless context clearly indicates otherwise.

To aid the Patent Office and any readers of any patent issued on this application in interpreting the claims appended hereto, Applicant that it does not intend any of the claims or claim elements to invoke 35 U.S.C. 112 (f) unless the words “means for” or “step for” are explicitly used in the particular claim.

While the present teachings have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings. In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

What is claimed is:

1. A system for managing treatment of a patient comprising:
a computer;
a database in data communication with said computer, said database having a plurality of prompts, a plurality of medical content, and a plurality of patient medical history;
a user device in data communication with said computer for presenting a first prompt received from said computer to the patient;
a life monitoring system in data communication with said computer, said life monitoring system observing aspects of the patient;
said user device receiving a first patient reaction to the first prompt, said first patient reaction being at least one of a text response, a selection, a voice response, and a patient biometric data from said life monitoring system;
software executing on said computer for receiving the first patient reaction, said database also receiving the first patient reaction;
(1) software executing on said computer configured with a generative artificial intelligence program, said software parsing the first patient reaction to generate a first key data set;
(2) software executing on said computer configured with a determinative artificial intelligence program, said software receiving the first key data set and accessing the database and associating one or more elements of the first key data set with one or more of the plurality of prompts and the plurality of medical content in said database, said software generating a first determinative data set;
(3) software executing on said computer configured with the generative artificial intelligence program receiving the first determinative data set, and said software generating a second prompt associated with the medical content associated with one or more elements of the key data set in (2);
the user device in data communication with said computer presenting the second prompt to the patient.

2. The system of claim 1, wherein the first prompt is in the form of at least one of an audio prompt and a visual prompt.

3. The system of claim 1, further comprising software executing on said computer for training a machine learning algorithm based on at least one of the plurality of patient medical history, the plurality of medical content, and any patient reactions provided to the system.

4. The system of claim 1, wherein the software executing on the computer configured with the determinative artificial intelligence program identifies discrete portions of the first patient reaction and is configured to receive the discrete portions of the first patient reaction before the first patient reaction is parsed by the software executing on said computer configured with the generative artificial intelligence program.

5. The system of claim 4, wherein each of the discrete portions of the first patient reaction not parsed by the generative artificial intelligence program is an image, text, or audio response.

6. The system of claim 1, wherein software executing on the computer is configured to determine relevant medical data associated with the first patient reaction, and the software executing on said computer configured with the generative artificial intelligence program modifies the second prompt and associated medical content in response to the relevant medical data.

7. The system of claim 1, further comprising software executing on said computer for generating an emergency alert in response to at least one of the first patient reaction and data from the life monitoring system.

8. The system of claim 1, wherein the life monitoring system collects data on a measurable vital aspect related to life or health of the patient.

9. The system of claim 1, wherein the software executing on said computer configured with the generative artificial intelligence program is configured to introduce variance into the second prompt.

10. The system of claim 1, wherein the software executing on said computer configured with the determinative artificial intelligence program is configured to introduce no variance when associating the one or more elements of the first key data set with the one or more of the plurality of prompts and medical content in the database, thereby generating substantially the same determinative data set from the same patient reaction.

11. The system of claim 10, wherein the patient medical history in the database includes patient reactions and system generated prompts from prior visits, wherein the software executing on the computer configured with the generative artificial intelligence program associates historical patient reactions to historical system generated prompts, and wherein variance is introduced into the first and second prompts to elicit improved patient reactions.

12. A system for managing treatment of a patient comprising:
a computer;
a database in data communication with said computer, said database having a plurality of prompts, a plurality of medical content, and a plurality of patient medical history;
a user device in data communication with said computer for presenting a first prompt received from said computer to the patient;
said user device receiving a first patient reaction to the first prompt, said first patient reaction being at least one of a text response, a selection, and a voice response;
software executing on said computer for receiving the first patient reaction, said database also receiving the first patient reaction;
(1) software executing on said computer configured with a generative artificial intelligence program, said software parsing the first patient reaction to generate a first key data set;
(2) software executing on said computer configured with a determinative artificial intelligence program, said software receiving the first key data set and accessing the database and associating one or more elements of the first key data set with one or more of the plurality of prompts and the plurality of medical content in said database, said software generating a first determinative data set;
(3) software executing on said computer configured with the generative artificial intelligence program receiving the first determinative data set, and said software generating a second prompt associated with the medical content associated with one or more elements of the key data set in (2);
the user device in data communication with said computer presenting the second prompt to the patient.

13. The system of claim 12, further comprising a life monitoring system in data communication with the computer, said life monitoring system observing aspects of the patient, wherein the first patient reaction received by the user device further includes patient biometric data from said life monitoring system.
